(19) United States
(12) Patent Application Publication
(10) Pub. No.: US 2022/0139503 A1
(43) Pub. Date: May 5, 2022

(54) DATA EXTRACTION FOR BIOPHARMACEUTICAL ANALYSIS

(71) Applicant: Tanvex BioPharma USA, Inc., San Diego, CA (US)

(72) Inventor: Kevin Yuen Fung Tse, San Diego, CA (US)

(21) Appl. No.: 17/428,944

(22) PCT Filed: Feb. 7, 2020
(86) PCT No.: PCT/US2020/017145

§ 371 Date: Aug. 5, 2021

Related U.S. Application Data
(60) Provisional application No. 62/803,339, filed on Feb. 8, 2019.

(51) Int. Cl. G16C 20/20; G16B 50/30; G16C 20/90; G01N 30/72

(52) U.S. Cl. CPC G16C 20/20; G01N 30/7233; G16C 20/90; G16B 50/30

(57) ABSTRACT

A method for extracting data for biopharmaceutical analysis may include selecting, based on a first path associated with a source directory, a first file included in the source directory. The first file may be parsed to identify, based on a reference mass value, one or more entries included in the first file. The one or more entries may each include a mass value. The one or more entries may be identified based on a difference between the mass value and the reference mass value being less than a threshold value. The one or more entries may be inserted into a second file.

### CROSS REFERENCE TO RELATED APPLICATION

This application claims priority to U.S. Provisional Application No. 62/803,339, which is filed on Feb. 8, 2019 and entitled “DATA EXTRACTION FOR BIOPHARMACEUTICAL ANALYSIS,” the disclosure of which is incorporated herein by reference in its entirety.

### TECHNICAL FIELD

The subject matter described herein relates generally to analytical chemistry and more specifically to the biopharmaceutical analysis.

### BACKGROUND

Biologics or biopharmaceuticals may refer to drugs that are based on large molecule proteins having a therapeutic effect. In order to ensure the pharmacokinetics/pharmacodynamics (PK/PD) and efficacy of a biopharmaceutical, biopharmaceutical characterization may be an integral part of drug development and manufacturing. Biopharmaceutical characterization may require identifying different species of molecules present in a biopharmaceutical including, for example, intact proteins, subunit proteins, peptides, glycans, and/or the like. For instance, a glycan may be a polysaccharide commonly found bonded to certain amino acid residues in a complex biologic protein. As such, the characterization of a biopharmaceutical may include analyzing the different glycans present in the biopharmaceutical. However, the analysis of the glycans present in the biopharmaceutical may require analyzing massive quantities of data.

### SUMMARY

Systems, methods, and articles of manufacture, including computer program products, are provided for extracting data for biopharmaceutical analysis. In one aspect, there is provided a system that includes at least one data processor and at least one memory. The at least one memory may store instructions that result in operations when executed by the at least one data processor. The operations may include: selecting, based at least on a first path associated with a source directory, a first file included in the source directory; parsing the first file to at least identify, based at least on a reference mass value, a first data entry included in the first file, the first data entry including a first mass value, and the first data entry being identified based at least on a difference between the first mass value and the reference mass value being less than a threshold value; and inserting, into a second file, the first data entry.

In some variations, one or more features disclosed herein including the following features may optionally be included in any feasible combination. The first data entry may further include an abundance value of a species having the first mass value.

In some variations, the species may be an intact protein, a subunit protein, a peptide, and/or a glycan.

In some variations, the first file may include a table. The first data entry may be stored in a row of the table. The first mass value may be stored in a first column of the table. The abundance value may be stored in a second column of the table.

In some variations, the first file may be an output from a mass spectrometer.

In some variations, the first file may be an Excel file and/or a portable document format (PDF) file generated by processing an output of a mass spectrometer.

In some variations, the second file included in the destination directory may be identified based at least on a second path associated with a destination directory.

In some variations, a third file may be selected based at least on the first path associated with the source directory. The third file may be parsed to at least identify, based at least on the reference mass value, a second data entry included in the third file. The second data entry may include a second mass value. The second data entry may be identified based at least on a difference between the second mass value and the reference mass value being less than the threshold value. The second data entry may be inserted into the second file. The third file may be selected in response to determining that the source directory includes one or more files in addition to the first file.

In some variations, the first data entry may be identified based at least on a first delimiter preceding the first data entry and/or a second delimiter succeeding the first data entry.

In another aspect, there is provided a method for extracting data for biopharmaceutical analysis. The method may include: selecting, based at least on a first path associated with a source directory, a first file included in the source directory; parsing the first file to at least identify, based at least on a reference mass value, a first data entry included in the first file, the first data entry including a first mass value, and the first data entry being identified based at least on a difference between the first mass value and the reference mass value being less than a threshold value; and inserting, into a second file, the first data entry.

In some variations, one or more features disclosed herein including the following features may optionally be included in any feasible combination. The first data entry may further include an abundance value of a species having the first mass value.

In some variations, the species may be an intact protein, a subunit protein, a peptide, and/or a glycan.

In some variations, the first file may include a table. The first data entry may be stored in a row of the table. The first mass value may be stored in a first column of the table. The abundance value may be stored in a second column of the table.

In some variations, the first file may be an output from a mass spectrometer.

In some variations, the first file may be an Excel file and/or a portable document format (PDF) file generated by processing an output of a mass spectrometer.

In some variations, the method may further include identifying, based at least on a second path associated with a destination directory, the second file included in the destination directory.

In some variations, the method may further include: selecting, based at least on the first path associated with the source directory, a third file included in the source directory; parsing the third file to at least identify, based at least on the reference mass value, a second data entry included in the third file, the second data entry including a second mass value, and the second data entry being identified based at least on a difference between the second mass value and the reference mass value being less than the threshold value; and inserting, into the second file, the second data entry.

In some variations, the first data entry may be identified based at least on a first delimiter preceding the first data entry and/or a second delimiter succeeding the first data entry.

In another aspect, there is provided a computer program product including a non-transitory computer readable medium storing instructions. The instructions may cause operations when executed by at least one data processor. The operations may include: selecting, based at least on a first path associated with a source directory, a first file included in the source directory; parsing the first file to at least identify, based at least on a reference mass value, a first data entry included in the first file, the first data entry including a first mass value, and the first data entry being identified based at least on a difference between the first mass value and the reference mass value being less than a threshold value; and inserting, into a second file, the first data entry.

In another aspect, there is provided a system that includes at least one data processor and at least one memory. The at least one memory may store instructions that result in operations when executed by the at least one data processor. The operations may include: selecting, based at least on a first path associated with a source directory, a first file included in the source directory; parsing the first file to at least identify a first data entry including a first peak value of a variant of a target biopharmaceutical; and inserting, into a second file, the first data entry.

In some variations, one or more features disclosed herein including the following features may optionally be included in any feasible combination. The first peak value may include at least one of a peak area, a peak retention time, and a percentage relative peak area.

In some variations, the variant may be a charge variant, a hydrophobic variant, or a size variant.

In some variations, the first file may be a chromatogram.

In some variations, the first data entry may be identified based at least on the first peak value exceeding a threshold value and/or being within a range of values.

In some variations, a type of the variant of the target biopharmaceutical may be identified based at least on the first data entry included in the second file.

In some variations, the variant may be identified as an acidic variant of the target biopharmaceutical based at least on the first peak value eluting earlier than a peak value of the target biopharmaceutical. The variant may be identified as a basic variant of the target biopharmaceutical based at least on the first target value eluting later than the peak value of the target biopharmaceutical.

In some variations, the variant may be identified, based at least on a first peak retention time of the variant and a second peak retention time of the target biopharmaceutical, as being more hydrophobic than the target biopharmaceutical or less hydrophobic than the target biopharmaceutical.

In some variations, a third file included in the source directory may be selected based at least on the first path associated with the source directory. The third file may be parsed to at least identify a second data entry including a second peak value of the variant of the target biopharmaceutical. The second data entry may be inserted into the second file. The first file may be a first chromatogram obtained at a first time. The second file may be a second chromatogram obtained at a second time. A sample of a biopharmaceutical including the variant is exposed to a first stress at the first time and a second stress at the second time. The first stress may be determined to produce a larger quantity of the variant than the second stress based at least on the second file.

In another aspect, there is provided a method. The method may include: selecting, based at least on a first path associated with a source directory, a first file included in the source directory; parsing the first file to at least identify a first data entry including a first peak value of a variant of a target biopharmaceutical; and inserting, into a second file, the first data entry.

In some variations, one or more features disclosed herein including the following features may optionally be included in any feasible combination. The first peak value may include at least one of a peak area, a peak retention time, and a percentage relative peak area.

In some variations, the variant may be a charge variant, a hydrophobic variant, or a size variant.

In some variations, the first file may be a chromatogram.

In some variations, the first data entry may be identified based at least on the first peak value exceeding a threshold value and/or being within a range of values.

In some variations, the method may further include identifying, based at least on the first data entry included in the second file, a type of the variant of the target biopharmaceutical.

In some variations, the variant may be identified as an acidic variant of the target biopharmaceutical based at least on the first peak value eluting earlier than a peak value of the target biopharmaceutical. The variant may be identified as a basic variant of the target biopharmaceutical based at least on the first target value eluting later than the peak value of the target biopharmaceutical.

In some variations, the variant may be identified, based at least on a first peak retention time of the variant and a second peak retention time of the target biopharmaceutical, as being more hydrophobic than the target biopharmaceutical or less hydrophobic than the target biopharmaceutical.

In some variations, the method may further include: selecting, based at least on the first path associated with the source directory, a third file included in the source directory; parsing the third file to at least identify a second data entry including a second peak value of the variant of the target biopharmaceutical; and inserting, into the second file, the second data entry. The first file may be a first chromatogram obtained at a first time. The second file may be a second chromatogram obtained at a second time. A sample of a biopharmaceutical including the variant is exposed to a first stress at the first time and a second stress at the second time. The first stress may be determined to produce a larger quantity of the variant than the second stress based at least on the second file.

In another aspect, there is provided a computer program product including a non-transitory computer readable medium storing instructions. The instructions may cause operations when executed by at least one data processor. The operations may include: selecting, based at least on a path associated with a source directory, a first file comprising a chromatograph included in the source directory; parsing the first file to at least identify a data entry including a peak value of a variant of a target biopharmaceutical; and inserting, into a second file, the data entry.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to data extraction for biopharmaceutical analysis, it should be readily understood that such features are not intended to be limiting. The claims that follow this disclosure are intended to define the scope of the protected subject matter.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating a biopharmaceutical analysis system, in accordance with some example embodiments;

FIG. 2A depicts an example a raw output file from a mass spectrometer, in accordance with some example embodiments;

FIG. 2B depicts another example a raw output file from a mass spectrometer, in accordance with some example embodiments;

FIG. 3A depicts an example of a processed output file from a mass spectrometer, in accordance with some example embodiments;

FIG. 3B depicts an example of a consolidated file, in accordance with some example embodiments;

FIG. 4 depicts a flowchart illustrating a process for extracting data for biopharmaceutical analysis, in accordance with some example embodiments; and

FIG. 5 depicts a block diagram illustrating a computing system, in accordance with some example embodiments.

When practical, similar reference numbers denote similar structures, features, or elements.

### DETAILED DESCRIPTION

Mass spectrometry such as, for example, liquid chromatography mass spectrometry (LC-MS), may be used to analyze the one or more species of molecules in a biopharmaceutical including, for example, intact proteins, subunit proteins, peptides, glycans, and/or the like. For example, mass spectrometry may be used to determine the relative abundance of different glycans present in one or more samples of the biopharmaceutical. The result of the mass spectrometry may include an individual output file for each sample of the biopharmaceutical. For instance, raw output files from a mass spectrometer may be processed to generate one processed output file for each sample of the biopharmaceutical. Because biopharmaceutical characterization is typically performed on numerous samples of the biopharmaceutical (e.g., upwards of 50), the subsequent analysis may span a large quantity of processed output files. As such, in some example embodiments, an analysis controller may be configured to extract, from multiple processed output files, one or more data entries with mass values matching a reference mass value of a molecule of interest. Furthermore, the analysis controller may be configured to insert, into a single consolidated file, the data entries extracted from the processed output files.

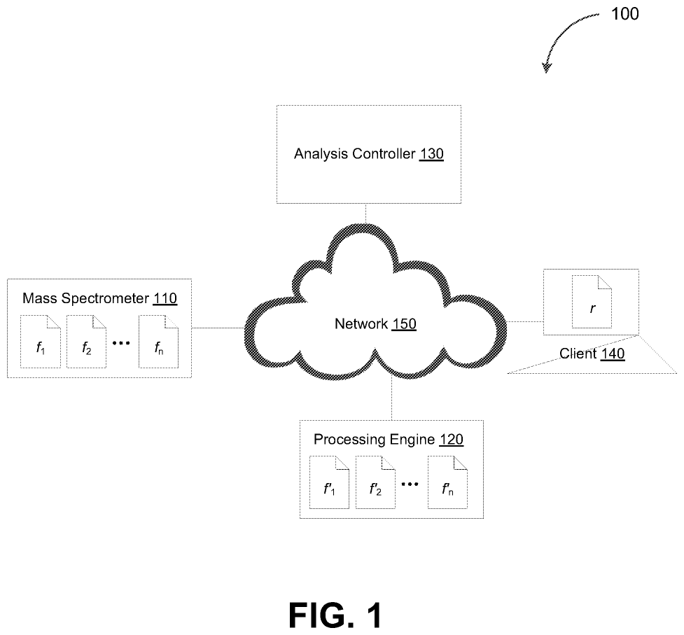

FIG. 1 depicts a system diagram illustrating a biopharmaceutical analysis system 100, in accordance with some example embodiments. Referring to FIG. 1, the biopharmaceutical analysis system 100 may include a mass spectrometer 110, a processing engine 120, an analysis controller 130, and a client 140. As shown in FIG. 1, the mass spectrometer 110, the processing engine 120, the analysis controller 130, and the client 140 may be communicatively coupled via a network 150. The network 150 may be any wired and/or wireless network including, for example, a local area network (LAN), a virtual local area network (VLAN), a wide area network (WAN), a public land mobile network (PLMN), the Internet, and/or the like. Meanwhile, the client 140 may be any processor-based device including, for example, a workstation, a desktop computer, a laptop computer, a tablet computer, a mobile device, a wearable apparatus, and/or the like. FIG. 1 shows the processing engine 120 and the analysis controller 130 as being deployed remotely, for example, as cloud-based software, web applications, and/or the like. However, it should be appreciated that at least some of the functionalities associated with the processing engine 120 and/or the analysis controller 130 may also be implemented locally at the client 140. For instance, the analysis controller 130 may be implemented as a script (e.g., a Visual Basic for Applications (VBA) script and/or the like) such that the logic associated with the analysis controller 130 may be executed at the client 140 without requiring any compilation.

Table 1 below depicts pseudo programming code implementing the analysis controller 130.

TABLE 1
Set up variables String/Variant/Boolean/Integer/Double
Set up variables for excel Workbooks
Set up variables for excel Worksheets
Set up variables for visual Charts
Function Click_CommandButton {
“Initialize Global Commands”
Clear cells in Glycan Data Extraction excel
Initialize variables and assign folderPaths to text field inputs
If source and destination folderPaths are not found - send error
message path not found
Ask for input for new reportName - send error message if name already
exists in folder
If input reportName is empty - send error message empty field
Send messages to confirm directories and file names
Initialize projectType to OptionButton selected - send message to
confirm project
“Populate Header Information”
Record folderPath and reportName to header as text
Set up two tables side by side
Record mass shift numbers for species searched as text in table 1
Record column header information and species names as text in table 1
Record column header information and percent species names as text in
table 2 to the right
“Parse Through Files”
Count total number of files to process in folderPath with the
Function countFiles (see below)
Store all filePaths to listArray and number of files to fileCounter
Send message to confirm number of files in folderpath
For each filePath in listArray - iterate the Function
extractSampleData (see below) and continue until fileCounter is
reached
“Calculate Species Percentages”
Send message all data files are processed and percent species is
being calculated
Move excelPosition to table 2
For each file - calculate percent species based on table 1 relative
abundance and sum abundance values
Move excelPosition to iterate the calculation for all samples in
folderPath
Send message data extraction complete and report is being formatted
Set up manual formulas for troubleshooting at the bottom of table 1
“Format Report”
Set alignment, numberFormat, autoFit, columnWidth, colorIndex,
borderLinestyle, borderWeight
Set borders in excel headers and around each table
“Create 4 charts each to different relevant species in the figure”
For each of the 4 charts - locate the chart at the desired
excelPosition with the desired height, width, chartType, chartTitle,
axesTitles, seriesName, seriesColor
“Create Results Report”
Send message creating results report
Create new resultsReport with the name reportName + “Results” and
save
Copy table 1 original report to this new report
Format results for alignment, numberFormat, autofit, columnWidth,
colorIndex, borderLinestyle, borderWeight
Create 4 charts - locate the chart at the desired excelPosition with
the desired height, width, chartType, chartTitle, axesTitles,
seriesName, seriesColor
}
Function countFiles (folderPath) {
“Count the Number of Files in Folder”
Take folderPath as destination
While fileName is not empty - increase countFiles by 1 and go to next
fileName
Return countFiles
}
Function extractSampleData (filePath) {
“Extract Critical Species from Sample File to Set Mass Searching
Starting Point”
Set currentFile as filePath
Move excelPosition to table 1
Based on projectType, run Function searchForMass (projectTypeMass)
(see below) and set referenceMass as currentMass
“Extract All Relevant Species from Sample File”
For each of the relevant species, run Function searchForMass (mass
shift from referenceMass) (see below)
Record both the returned value from the Function and the currentMass
in table 1
Sum the relative abundance values of all the species
Close the currentFile
}
Function searchForMass (mass) {
“Search for Mass in Input File”
Set up variables Integer/Double
Initialize variables and define upper/lower limit for mass tolerance
Iterate for each species in the excel file - while the mass detected
in column 1 is not empty - if the value in column 1 is within the
tolerance of the mass desired - return this species relative
abundance and set currentMass to the mass detected

Referring again to FIG. 1, the mass spectrometer 110 may be configured to analyze one or more samples of a biopharmaceutical. For each sample of the biopharmaceutical, the mass spectrometer 110 may generate a spectrum corresponding to a relative abundance of one or more different species of molecules present in the sample of the biopharmaceutical including, for example, intact proteins, subunit proteins, peptides, glycans, and/or the like. The raw output of the mass spectrometer 110 may include a raw output file (e.g., a .raw file and/or the like) for each sample of the biopharmaceutical analyzed by the mass spectrometer 110. For example, for an n quantity of samples of the biopharmaceutical, the mass spectrometer 110 may generate an n quantity of raw output files (e.g., f₁, f₂, . . . , fₙ). Each of the n quantity of raw output files may correspond to one of the n quantity of samples of the biopharmaceutical.

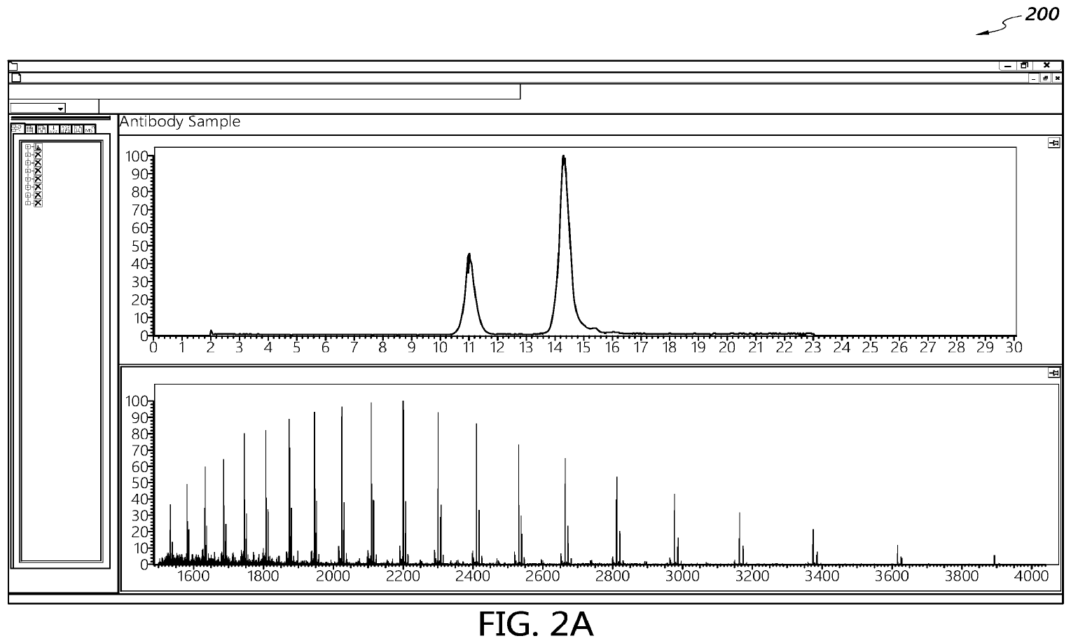

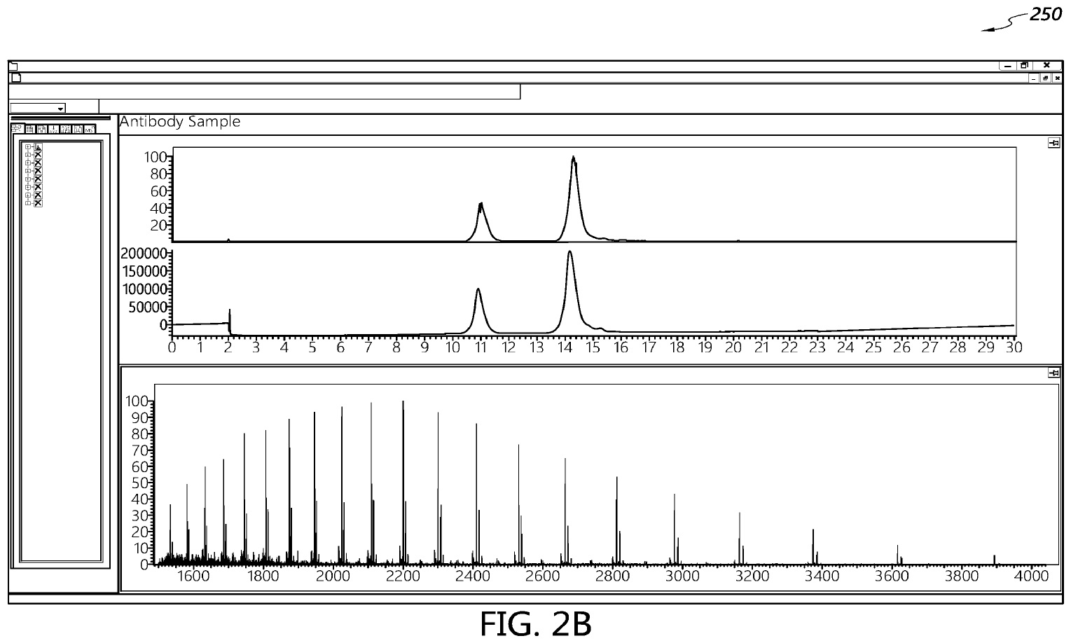

To further illustrate, FIGS. 2A-B depict examples of raw output files from the mass spectrometer 110, in accordance with some example embodiments. Referring to FIG. 2A, the mass spectrometer 110 may output a raw output file 200, which may include the results of analyzing a sample of a biopharmaceutical (e.g., an antibody). As shown in FIG. 2A, the results in the raw output file 200 may include the spectrum corresponding to the relative abundance values of different species of molecules present in the sample of the biopharmaceutical. For example, the top graph in the raw output file 200 may trace the time course of the raw data against the total ion current measured by the mass spectrometer 110. Based on a selection of a certain time window (e.g., retention time), the bottom graph in the raw output file 200 may display the corresponding raw data spectrum. This raw data spectrum may be a Fourier-transformed signal from the mass spectrometer 110 that charts the mass-to-charge (m/z) ratio of the ions detected to the relative abundance of those ions. Moreover, this raw data spectrum may be used to compute the relative abundance value of different species of molecules present in the sample of the biopharmaceutical.

Alternatively, the mass spectrometer 110 may also output a raw output file 250 as shown in FIG. 2B. In addition to the relative abundance values of the different species of molecules present in the sample of the biopharmaceutical, the raw output file 250 may include the ultraviolet absorbance values, visible absorbance values, and/or reflectance values of the sample of the biopharmaceutical. The raw output file 250 shown in FIG. 2B may be a raw output file having an alternate format than the raw output file 200 shown in FIG. 2A. The top graph in the raw output file 250 may trace the time course of the raw data against the total ion current as well as the ultraviolet absorbance detected at a certain wavelength. Meanwhile, the bottom graph in the raw output file 250 may display the same raw data spectrum as the raw output file 200 (e.g., a raw data spectrum for a certain time window).

The processing engine 120 may be configured to process the raw output files (e.g., .raw files and/or the like) from the mass spectrometer 110 and generate, for each raw output file, a corresponding processing output file (e.g., an Excel file, a Word file, a Portable Document File (PDF) file, and/or the like). For instance, as shown in FIG. 1, the processing engine 120 may generate, based on the n quantity of raw output files (e.g., f₁, f₂, . . . , fₙ), an n quantity of processed output files (e.g., f′₁, f′₂, . . . , f′ₙ). Each of the n quantity of processed output files may correspond to one of the n quantity of raw output files from the mass spectrometer 110. Moreover, the n quantity of processed output files may be stored in a source directory, for example, accessible to the analysis controller 130.

In some example embodiments, a processed output file may be generated to include an entry for each species of molecule (e.g., intact protein, subunit protein, peptide, glycan, and/or the like) present in a corresponding sample of the biopharmaceutical. The entry for a species of molecule may include a mass value and a relative abundance value for that species of molecule. Furthermore, successive data entries in the processed output file may be separated by one or more delimiters including, for example, whitespace characters, commas, colons, and/or the like. These delimiters may enable the analysis controller 130 to identify different data entries within the processed output file.

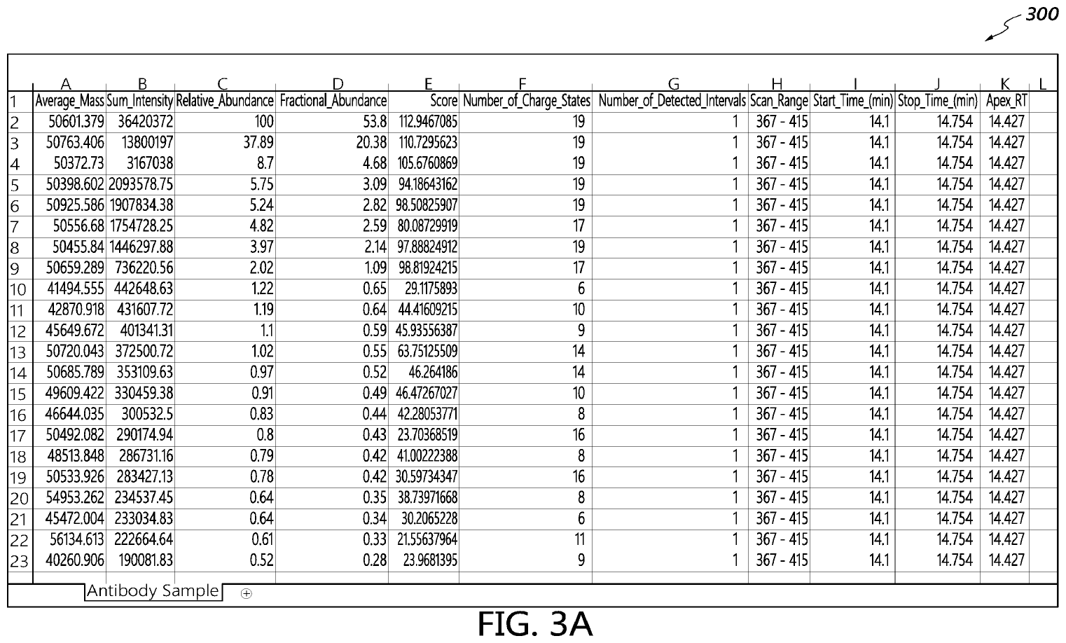

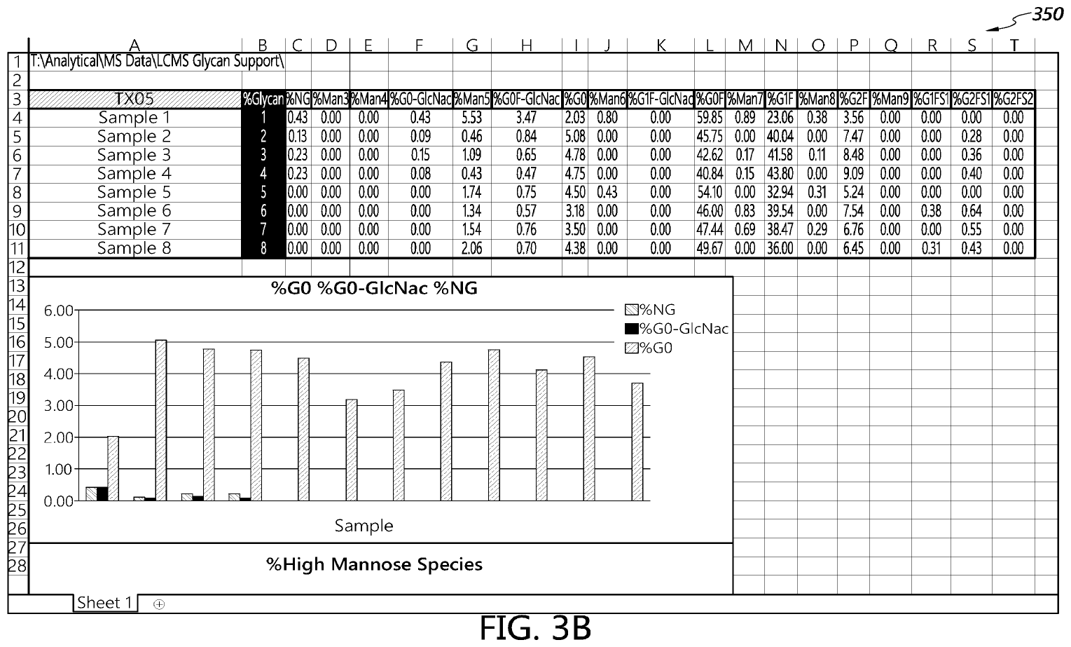

To further illustrate, FIG. 3A depicts an example of a processed output file 300 from the mass spectrometer 110, in accordance with some example embodiments. For instance, the processing engine 120 may generate the processed output file 300 based on the raw output file 200 and/or the raw output file 250. In the example shown in FIG. 3A, the processed output file 300 may be an Excel file. Accordingly, each row in the Excel spreadsheet shown in FIG. 3A may correspond to one entry in the processed output file 300. The mass values for the entries may be stored in one column of the spreadsheet whereas the relative abundance values for the entries may be stored in a different column of the spreadsheet. Nevertheless, it should be appreciated that the processed output file 300 may be any type of file in which the data entries in the processed output file 300 may be stored in a structured manner including, for example, a Word file, a Portable Document File (PDF) file, and/or the like.

In some example embodiments, the analysis controller 130 may be configured to generate, based at least on the n quantity of processed output files (e.g. f′₁, f′₂, . . . , f′ₙ), a single consolidated file r. The analysis controller 130 may generate the consolidated file r by at least extracting, from each of the n quantity of processed output files, one or more data entries with mass values that match a reference mass value of a molecule of interest. It should be appreciated that a mass value may be determined to match a reference mass value if a difference between the two mass values do not exceed a threshold value. For example, the analysis controller 130 may extract, from a first processed output file f′₁, a data entry based at least on the mass value of the data entry matching a reference mass value of a glycan of interest. That data entry, including the mass value and the relative abundance associated with the data entry, may be inserted into the consolidated file r.

FIG. 3B depicts an example of a consolidated file 350, in accordance with some example embodiments. As noted, in some example embodiments, the analysis controller 130 may generate the consolidated file r by at least inserting, into the consolidated file r, one or more data entries extracted from the n quantity of processed output files (e.g., f′₁, f′₂, . . . , f′ₙ). Accordingly, the consolidated file r may include one or more data presentation providing a visual representation of the data entries extracted from the n quantity of processed output files. For instance, in the example shown in FIG. 3B, the consolidated file r may include a table as well as a chart, each of which providing a different visual representation of the data entries extracted from the n quantity of processed output files. The analysis controller 130 may store the consolidated file r in a destination directory, where the consolidated file r may be accessible to the client 140. As such, the consolidated file r may be displayed at the client 140 to enable a biopharmaceutical characterization of a corresponding biopharmaceutical based on the relative abundances of different species of molecules (intact proteins, subunit proteins, peptides, glycans, and/or the like) present in the biopharmaceutical.

In some example embodiments, instead of and/or in addition to identifying various species of molecules that may be present in a biopharmaceutical based on processed output files from the mass spectrometer 110, the analysis controller 130 may parse chromatograms output by a liquid chromatography instrument to identify one or more variants of the biopharmaceutical. As used herein, a variant may refer to a molecule of a biopharmaceutical having one or more structural differences when compared to a target biopharmaceutical including, for example, the presence of an additional functional group, an oxidized amino acid, and/or the like. These structural differences may further manifest as differences in the characteristics of the variants including, for example, charge, hydrophobicity, size, and/or the like. Accordingly, examples of variants may include charge variants (e.g., acidic variants, basic variants, and/or the like), hydrophobic variants (e.g., variants that are more or less hydrophobic than the target biopharmaceutical), and size variants (e.g., aggregates, oligomers, dimers, monomers, and/or the like).

Detecting the presence of variants may be akin to detecting the presence of impurities in the biopharmaceutical. For example, the presence of variants may be detected by examining intact protein molecules to identify those intact protein molecules that are structurally different than the intact protein molecules associated with the target biopharmaceutical. Depending on the type of structural differences exhibited by a variant, the presence of variants in the biopharmaceutical may have no effects or no adverse effects on the safety and performance of the biopharmaceutical. Nevertheless, the detection of impurities may be necessary during the manufacturing of the biopharmaceutical. Moreover, it may be desirable to keep the quantity of impurities present in the biopharmaceutical at a minimum or below a threshold level.

As noted, the analysis controller 130 may identify the variants that are present in the biopharmaceutical based on chromatograms output by a liquid chromatography instrument. The chromatograms associated with the biopharmaceutical may include, for each variant included in the biopharmaceutical, one or more peak values including, for example, a peak area, a peak retention time, a percentage relative peak area, and/or the like. Accordingly, the analysis controller 130 may parse one or more chromatograms in order to extract, from each chromatogram, the peak values associated with the target biopharmaceutical as well as the variants of the target biopharmaceutical. Moreover, the analysis controller 130 may insert, into a separate file, the peak values extracted from each chromatogram. For example, the analysis controller 130 may extract peak values that exceed one or more threshold values and/or are within one or more ranges of values. The resulting file may therefore include one or more peak values for the target biopharmaceutical as well as the variants of the target biopharmaceutical including, for example, peak values (e.g., peak area, peak retention time, and/or percentage relative peak area) for the target biopharmaceutical, charge variants of the target biopharmaceutical, size variants of the target biopharmaceutical, hydrophobic variants of the target biopharmaceutical, and/or the like.

In some example embodiments, the analysis controller 130 may extract peak values in order to further identify specific types of variants. For example, a variant may be identified, based at least in part on its peak retention time relative to the peak retention time of the target biopharmaceutical (e.g., when the variant elutes relative to the target biopharmaceutical), as being more hydrophobic than the target biopharmaceutical or less hydrophobic than the target biopharmaceutical. Alternatively and/or additionally, a variant may be identified, based at least in part on its peak retention time relative to the peak retention time of the target biopharmaceutical, as being a more acidic than the target biopharmaceutical or less acidic than the target biopharmaceutical. For instance, a variant with peak values that elute earlier than the peak values of the target biopharmaceutical may be a more acidic variant of the target pharmaceutical while a variant with peak values that elute later than the peak values of the target biopharmaceutical may be a more basic variant of the target pharmaceutical.

In some example embodiments, the analysis controller 130 may extract and analyze the peak values of variants across multiple chromatograms (e.g., of different samples of the biopharmaceutical). For example, the analysis controller 130 may extract and analyze peak values from a series of chromatograms, each of which associated with a same sample of a biopharmaceutical at different points in time. The sample of the biopharmaceutical may exhibit change and growth over time as the sample of the biopharmaceutical is exposed to different types of stresses such as, for example, a first stress at a first time, a second stress at a second time, and/or the like. Accordingly, the peak values that are included a first chromatogram obtained at the first time may be compared to the peak values that are included in a second chromatogram obtained at the second time in order to identify correlations between different types of stress and the various types of variants that may be present in the sample of the biopharmaceutical. For instance, peak values from different chromatograms may be compared in order to determine whether a certain type of stress produced a larger (or smaller) quantity of an acidic variant of the target biopharmaceutical, a basic variant of the target biopharmaceutical, a more hydrophobic variant of the target biopharmaceutical, a less hydrophobic variant of the target biopharmaceutical, and/or the like.

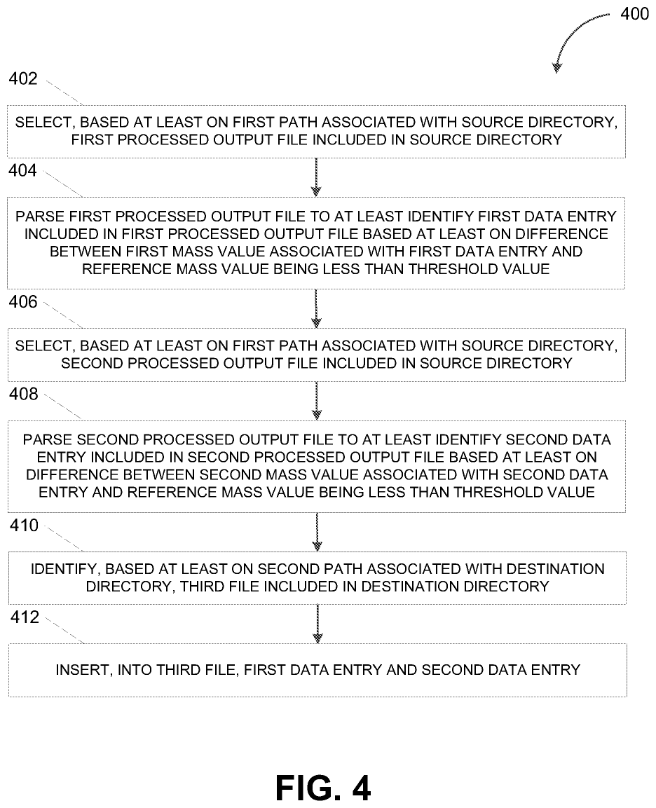

FIG. 4 depicts a flowchart illustrating a process 400 for extracting data for biopharmaceutical analysis, in accordance with some example embodiments. Referring to FIGS. 1, 2A-B, 3A-B, and 4, the process 400 may be performed by the analysis controller 130.

The analysis controller 130 may select, based at least on a first path associated with a source directory, a first processed output file included in the source directory (402). For example, the analysis controller 130 may select the first processed output file f′₁ based at least on the path of the source directory storing the n quantity of processed output files (e.g., f′₁, f′₂, . . . , f′ₙ).

The analysis controller 130 may parse the first processed output file to at least identify a first data entry included in the first processed output file based at least on a difference between a first mass value associated with the first data entry and a reference mass value being less than a threshold value (404). For example, the analysis controller 130 may identify a data entry in the first processed output file f′₁ based at least on the data entry being associated with a mass value that matches a reference mass value of a molecule of interest. In some example embodiments, the mass value associated with the data entry may be determined to match the reference mass value if the difference between the two mass values do not exceed a threshold value.

The analysis controller 130 may select, based at least on the first path associated with the source directory, a second processed output file included in the source directory (406). For example, the analysis controller 130 may select a second processed output file f′₂ based at least on the path of the source directory storing the n quantity of processed output files (e.g., f′₁, f′₂, . . . , f′ₙ). It should be appreciated that the analysis controller 130 may select additional processed output files from the source directory until the analysis controller 130 has parsed all n quantity of processed output files (e.g., f′₁, f′₂, . . . , f′ₙ) in the source directory.

The analysis controller 130 may parse the second processed output file to at least identify a second data entry included in the second processed output file based at least on a difference between a second mass value associated with the second data entry and the reference mass value being less than the threshold value (408). For example, the analysis controller 130 may identify a data entry in the second processed output file f′₂ based at least on the data entry being associated with a mass value that matches the reference mass value of a molecule of interest. As noted, the mass value associated with the data entry may be determined to match the reference mass value if the difference between the two mass values do not exceed a threshold value.

The analysis controller 130 may identify, based at least on a second path associated with a destination directory, a third file included in the destination directory (410). For example, the analysis controller 130 may identify the consolidated file r stored in a destination directory based at least on a path of the destination directory.

The analysis controller 130 may insert, into the third file, the first data entry and the second data entry (412). For example, the analysis controller 130 may insert, into the consolidated file r, the data entries identified in operations 404 and 408. In doing so, the analysis controller 130 may generate the consolidated file r to include the relative abundance values of the different species of molecules (intact proteins, subunit proteins, peptides, glycans, and/or the like) that are present in multiple samples of the biopharmaceutical. As FIG. 3B shows, the consolidated file r may include one or more data presentation providing a visual representation of the data entries extracted from the n quantity of processed output files. Moreover, the consolidated file r may be displayed at the client 140 to enable a biopharmaceutical characterization of a corresponding biopharmaceutical based on the relative abundances of different species of molecules (intact proteins, subunit proteins, peptides, glycans, and/or the like) present in the biopharmaceutical.

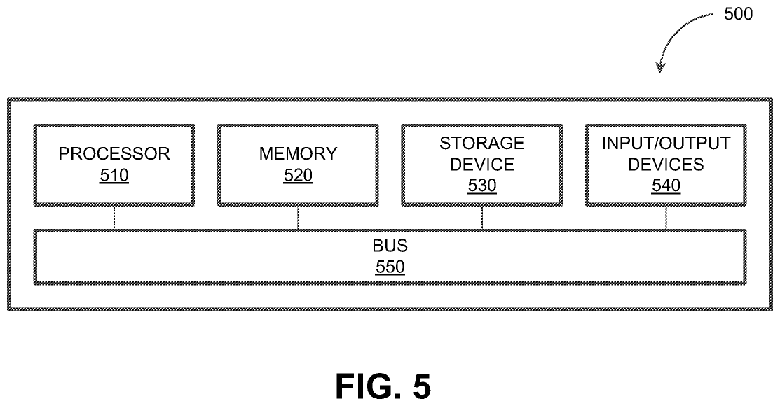

FIG. 5 depicts a block diagram illustrating a computing system 500 consistent with implementations of the current subject matter. Referring to FIGS. 1 and 5, the computing system 500 can be used to implement the mass spectrometer 110, the processing engine 120, the analysis controller 130, the client 140, and/or any components therein.

As shown in FIG. 5, the computing system 500 can include a processor 510, a memory 520, a storage device 530, and an input/output device 540. The processor 510, the memory 520, the storage device 530, and the input/output device 540 can be interconnected via a system bus 550. The processor 510 is capable of processing instructions for execution within the computing system 500. Such executed instructions can implement one or more components of, for example, the mass spectrometer 110, the processing engine 120, the analysis controller 130, the client 140. In some example embodiments, the processor 510 can be a single-threaded processor. Alternately, the processor 510 can be a multi-threaded processor. The processor 510 is capable of processing instructions stored in the memory 520 and/or on the storage device 530 to display graphical information for a user interface provided via the input/output device 540.

The memory 520 is a computer readable medium such as volatile or non-volatile that stores information within the computing system 500. The memory 520 can store data structures representing configuration object databases, for example. The storage device 530 is capable of providing persistent storage for the computing system 500. The storage device 530 can be a floppy disk device, a hard disk device, an optical disk device, a tape device, a solid-state device, and/or other suitable persistent storage means. The input/output device 540 provides input/output operations for the computing system 500. In some example embodiments, the input/output device 540 includes a keyboard and/or pointing device. In various implementations, the input/output device 540 includes a display unit for displaying graphical user interfaces.

According to some example embodiments, the input/output device 540 can provide input/output operations for a network device. For example, the input/output device 540 can include Ethernet ports or other networking ports to communicate with one or more wired and/or wireless networks (e.g., a local area network (LAN), a wide area network (WAN), the Internet).

In some example embodiments, the computing system 500 can be used to execute various interactive computer software applications that can be used for organization, analysis and/or storage of data in various formats. Alternatively, the computing system 500 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 540. The user interface can be generated and presented to a user by the computing system 500 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random query memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A system, comprising:
at least one data processor; and
at least one memory storing instructions, which when executed by the at least one data processor, result in operations comprising:
selecting, based at least on a first path associated with a source directory, a first file included in the source directory;
parsing the first file to at least identify, based at least on a reference mass value, a first data entry included in the first file, the first data entry including a first mass value, and the first data entry being identified based at least on a difference between the first mass value and the reference mass value being less than a threshold value; and
inserting, into a second file, the first data entry.

2. The system of claim 1, wherein the first data entry further includes an abundance value of a species having the first mass value.

3. The system of claim 2, wherein the species comprises an intact protein, a subunit protein, a peptide, and/or a glycan.

4. The system of claim 2, wherein the first file includes a table, wherein the first data entry is stored in a row of the table, wherein the first mass value is stored in a first column of the table, and wherein the abundance value is stored in a second column of the table.

5. The system of claim 1, wherein the first file comprise an output from a mass spectrometer.

6. The system of claim 1, wherein the first file comprise an Excel file and/or a portable document format (PDF) file generated by processing an output of a mass spectrometer.

7. The system of claim 1, further comprising:
identifying, based at least on a second path associated with a destination directory, the second file included in the destination directory.

8. The system of claim 1, further comprising:
selecting, based at least on the first path associated with the source directory, a third file included in the source directory;
parsing the third file to at least identify, based at least on the reference mass value, a second data entry included in the third file, the second data entry including a second mass value, and the second data entry being identified based at least on a difference between the second mass value and the reference mass value being less than the threshold value; and
inserting, into the second file, the second data entry.

9. The system of claim 8, wherein the third file is selected in response to determining that the source directory includes one or more files in addition to the first file.

10. The system of claim 1, wherein the first data entry is identified based at least on a first delimiter preceding the first data entry and/or a second delimiter succeeding the first data entry.

11. A computer implemented method, comprising:
selecting, based at least on a first path associated with a source directory, a first file included in the source directory;
parsing the first file to at least identify, based at least on a reference mass value, a first data entry included in the first file, the first data entry including a first mass value, and the first data entry being identified based at least on a difference between the first mass value and the reference mass value being less than a threshold value; and
inserting, into a second file, the first data entry.

12. The method of claim 11, wherein the first data entry further includes an abundance value of a species having the first mass value.

13. The method of claim 12, wherein the species comprises an intact protein, a subunit protein, a peptide, and/or a glycan.

14. The method of claim 12, wherein the first file includes a table, wherein the first data entry is stored in a row of the table, wherein the first mass value is stored in a first column of the table, and wherein the abundance value is stored in a second column of the table.

15. The method of claim 11, wherein the first file comprise an output from a mass spectrometer.

16. The method of claim 11, wherein the first file comprise an Excel file and/or a portable document format (PDF) file generated by processing an output of a mass spectrometer.

17. The method of claim 11, further comprising:
identifying, based at least on a second path associated with a destination directory, the second file included in the destination directory.

18. The method of claim 11, further comprising:
selecting, based at least on the first path associated with the source directory, a third file included in the source directory;
parsing the third file to at least identify, based at least on the reference mass value, a second data entry included in the third file, the second data entry including a second mass value, and the second data entry being identified based at least on a difference between the second mass value and the reference mass value being less than the threshold value; and
inserting, into the second file, the second data entry.

19. The method of claim 11, wherein the first data entry is identified based at least on a first delimiter preceding the first data entry and/or a second delimiter succeeding the first data entry.

20. A non-transitory computer readable medium storing instructions, which when executed by at least one data processor, result in operations comprising:
selecting, based at least on a first path associated with a source directory, a first file included in the source directory;
parsing the first file to at least identify, based at least on a reference mass value, a first data entry included in the first file, the first data entry including a first mass value, and the first data entry being identified based at least on a difference between the first mass value and the reference mass value being less than a threshold value; and
inserting, into a second file, the first data entry.

21-41. (canceled)
